(19) United States
(12) Patent Application Publication
(10) Pub. No.: US 2017/0095593 A1
(43) Pub. Date: Apr. 6, 2017

(54) ADIPOSE-DERIVED STEM CELL PRODUCT

(71) Applicant: Sharon Patricia McQuillan, Fort Lauderdale, FL (US)

(72) Inventor: Sharon Patricia McQuillan, Fort Lauderdale, FL (US)

(21) Appl. No.: 15/282,167

(22) Filed: Sep. 30, 2016

Related U.S. Application Data
(60) Provisional application No. 62/236,596, filed on Oct. 2, 2015.

(51) Int. Cl. A61L 27/36; A61L 27/50; A61M 1/00; C12N 5/0775; C12N 9/22; A61K 35/35; A61L 27/54

(52) U.S. Cl. CPC A61L 27/3687; A61K 35/35; A61L 27/3604; A61L 27/3645; A61L 27/50; A61L 27/54; C12N 5/0667; C12N 9/22; C12Y 301/21001; A61M 1/008; A61M 1/0068; A61M 1/0094; A61L 2300/64; A61L 2400/06; A61L 2300/412

(57) ABSTRACT

The present invention discloses adipose-derived stem cells (ADSCs), adipose-derived stem cell-enriched fractions (ADSC-EF), their compositions and kits containing the ADSC's and their enriched fractions, and enzyme blends useful for their isolation, as well as methods of their isolation and use.

### CROSS-REFERENCE TO A RELATED APPLICATION

This application claims the benefit of U.S. Provisional Application Ser. No. 62/236,596, filed Oct. 2, 2015, which is hereby incorporated by reference in its entirety, including any figures, tables, or drawings.

### FIELD OF THE DISCLOSURE

The invention relates to a mixed population of stem cells isolable from mammalian adipose tissue that may be differentiated into or regenerated into various mammalian tissues and, more particularly, to adipose-derived stem cell products, pharmaceutical compositions containing such stem cells that are, inter alia, useful for treatment of medical conditions and cosmetic indications.

### BACKGROUND OF THE DISCLOSURE

The identification of mesenchymal stem cells, chiefly obtained from bone marrow, has led to advances in tissue regrowth and differentiation. These pluripotent cells found in bone marrow and periosteum are capable of differentiating into various mesenchymal or connective tissues. It has been suggested that such cells are useful for repair of tissues such as cartilage, fat, and bone (see, e.g., U.S. Pat. Nos. 5,908,784, 5,906,934, 5,827,740, 5,827,735. While stem cell utilization continues to expand, both research and commercial application advances are being slowed by their concentration levels in donor tissue, the rate of variation in stem cell levels in different donors and variable and incomplete isolation methodologies. Alternative sources to stem cells such as adipose tissue have lessened some of the difficulties associated with bone marrow harvesting, including pain experienced by the donor. U.S. Pat. No. 6,200,606 by Peterson et al., describes the isolation of CD34+ bone or cartilage precursor cells (of mesodermal origin) from tissues, including adipose. U.S. Pat. No. 7,470,537 by Hedrick et al., describes certain adipose-derived stem cells, adipose-derived stem cell fractions, lattices, and methods for obtaining the cells, fractions, and lattices.

Adipose tissue has proven to be a rich source of cells that participate in the natural healing process, including endothelial progenitor cells, fibroblasts, pre-adipocytes, pecicytes, macropbages, and mesenchymal stem cells. Several methods to isolate certain of these cell populations have been described for clinical research including lecithin digestion, collagenase digestion, and various attempts at mechanical isolation. See Zuk, P. A. et al., Mol. Biol. Cell. 2002 December; 13(12): 4279-95; Miranville, A. et al., Circulation, 2004 . 20; 110(3): 349-55; Planat-Benard, V. et al., Circ Res. 2004 Febuary 6; 223-9; Zuk, P. A. et al., Tissue Eng. 2001 April; 7(2): 211-28; Hicok, K. C. et al., Tissue Eng. 2004 March-April; 10(3-4): 371-380; Erickson, G. R., Biochem. Res. Commun. 2003 Feb. 21; 301(4): 1016-22; Cousin, B. et al., Biochem. Biophys. Res. Commun. 2003 Feb. 21; 301(4): 1016-22; Safford, K. M. et al. Biochem. Biophys. Res. Commun. 2002 Jun. 7; 294(2): 371-9; Bourin, P. et al., Cytotherapy, 2013; 15: 641-648; Baptista, L. S. et al., Cytotherapy. 2009; 11(6): 706-715; Bunnell, B. A. et al., Methods, 2008 June; 45(2): 115-120; Chajchir, A. et al., Aesthetic Plastic Surgery. 1993; 17(2): 113-115; Domenis, R. et al., Stem Cell Research& Therapy, 2015; 6(2): epublication before print; Yoshimura, Kotaro et al., Published Patent Application US 2007/0148766; Paspaliaris, B. et al., Published Patent Application US 2014/0093482; Victor, S., Published Patent Application US 2013/0189234; Fraser, J. K. et al., Published Patent Application US 2010/0015104. These attempts have led to varying, uncharacterized, mixed populations of cells making clinical research difficult to conduct and/or interpret.

Even with the identification of adipose tissue as a stem cell source, there remains a need for a more readily purifiable, consistent stem cell product from adipose tissue that would improve or enhance use of the cells to treat a variety of pharmacological conditions, as well as enable more routine implantation for a number of cosmetic conditions. Methods that may more readily isolate a large percentage of the stem cells present in adipose tissue would also further their use in medical treatment or cosmetic implantation, especially when those isolates further contain other cells that participate in the natural healing process. Kits having the capability to standardize such methodology and provide the materials necessary to carry out standardized isolation techniques would simplify efforts for those administering the products and provide more consistent and predictable results.

It is a common problem for stem cell researchers to extract a large percentage of the available stem cells from an adipose tissue sample. First, the levels of available stem cells in a particular sample vary from donor to donor. The isolation method may need to be individually adjusted based on donor, as well as on the number of cells available. Such adjustments have the potential to lead to inconsistent cell products, necessitating the product's analysis before each use and complicating its administration. Second, digestion of the adipose tissue often leads to the generation of impurities which can require extensive purification to isolate the stem cells in usable form. Enzyme digestion may also result in the release of DNA, which can further associate with the stem cells, making them less accessible when employing prior art isolation methods.

Inasmuch as stem cell levels present in their isolated fractions or compositions, and in particular, in those derived from mammalian adipose tissue, remain variable, even when derived from a singular donor, there remains an unfulfilled need for standardization of digestion materials and methods for stem cell isolation. There is a further unmet need to provide these standardized stem cell products, materials and methods for use by administers of treatments or implantations of these products to provide more consistent predictable therapies in a recipient. The stem cell products, digestion enzyme blends, kits containing such products, blends compositions, and the like, as well as processes for isolation and methods of use disclosed herein are directed to these, as well as other important ends.

### SUMMARY OF THE DISCLOSURE

The present invention is generally directed to stem cells isolable from mammalian adipose tissue and, more particularly, to adipose-derived stem cell products, pharmaceutical compositions containing such stem cells that are, inter alia, useful for treatment of medical conditions and for cosmetic indications, as well as methods of their manufacture or use, enzyme compositions useful in their preparation, and kits for preparing stem cells or their compositions containing such enzyme compositions.

In one embodiment, the invention is directed to stem cell compositions comprising: a population of stem cells obtained from mammalian adipose tissue; said population of stem cells comprising: endothelial progenitor cells, red blood cells, fibroblasts, white blood cells, hematopoietic stem cells and mesenchymal stem cells; wherein the stem cell mixture contains less than about 0.5 EU/cc endotoxin; and wherein the stem cell mixture is at least about 95% viable as determined by flow cytometry.

In another embodiment, the invention is directed to enzyme compositions comprising: a proteolytic enzyme capable of digesting adipose tissue; and an enzyme that catalyzes the hydrolytic cleavage of phosphodiester linkages in DNA; wherein the enzyme composition used for the dissociation of said adipose tissue contains from about 0.25 to about 0.75 Wunsch units or from about 250 to about 750 Collagen Digestion Units and from about 7 to about 15 Kunitz units of DNase I per cubic centimeter of adipose tissue.

In certain embodiments, the invention is directed to enzyme compositions comprising: a proteolytic enzyme capable of digesting adipose tissue; and an enzyme that catalyzes the hydrolytic cleavage of phosphodiester linkages in DNA; wherein said enzyme composition is sufficient to digest said human adipose tissue, and provide a stem cell composition containing at least about 2.0E+05 cells per cc lipoaspirate.

In still another embodiment, the invention is directed to pharmaceutical kits for isolation of stem cells from mammalian adipose tissue comprising a container having an effective amount of an adipose digestion enzyme composition comprising: a proteolytic enzyme capable of digesting adipose tissue; and an enzyme that catalyzes the hydrolytic cleavage of phosphodiester linkages in DNA; wherein the enzyme composition used for the dissociation of said adipose tissue contains from about 0.25 to about 0.75 Wunsch units or from about 250 to about 750 Collagen Digestion Units and from about 7 to about 15 Kunitz units of DNase I per cubic centimeter of adipose tissue.

In yet another embodiment, the invention is directed to pharmaceutical kits for isolation of stem cells from mammalian adipose tissue comprising a container having an effective amount of an adipose digestion enzyme composition comprising: a proteolytic enzyme capable of digesting adipose tissue; and an enzyme that catalyzes the hydrolytic cleavage of phosphodiester linkages in DNA; wherein said enzyme composition is sufficient to digest said human adipose tissue, and provide a stem cell composition containing at least about 2.0E+05 cells per cc LPA.

In another embodiment, the invention is directed to methods for isolating stem cells from mammalian adipose tissue comprising the steps of: contacting mammalian adipose tissue with an enzyme composition comprising: a proteolytic enzyme capable of digesting adipose tissue; and an enzyme that catalyzes the hydrolytic cleavage of phosphodiester linkages in DNA; wherein the enzyme composition used for the dissociation of said adipose tissue contains from about 0.25 to about 0.75 Wunsch units or from about 250 to about 750 Collagen Digestion Units and from about 7 to about 15 Kunitz units of DNase I per cubic centimeter of adipose tissue; for a time and under conditions effective to digest the adipose tissue; and isolating the cells from the enzyme digested adipose tissue; wherein the adipose tissue was extracted from a mammal.

In another embodiment, the invention is directed to methods for isolating stem cells from mammalian adipose tissue comprising the steps of: contacting mammalian adipose tissue with an enzyme composition comprising: a proteolytic enzyme capable of digesting adipose tissue; and an enzyme that catalyzes the hydrolytic cleavage of phosphodiester linkages in DNA; wherein said enzyme composition is sufficient to digest said human adipose tissue, and provide a stem cell composition containing at least about 2.0E+05 cells per cc lipoaspirate; for a time and under conditions effective to digest the adipose tissue; and isolating the cells from the enzyme digested adipose tissue; wherein the adipose tissue was extracted from a mammal.

In yet another embodiment, the invention is directed to methods for treating a medical condition amenable to treatment with stem cells, comprising the steps of: isolating stem cells from adipose tissue of a patient in need of treatment for a medical condition amenable to treatment with stem cells; providing a stem cell composition derived from said isolated stem cells; and re-implanting an effective amount of said stem cell composition into an anatomical region of-said patient, wherein said region is affected with said medical condition, wherein said stem cell composition comprises: a population of stem cells obtained from mammalian adipose tissue; said population of stem cells comprising: endothelial progenitor cells, red blood cells, fibroblasts, white blood cells, hematopoietic stem cells and mesenchymal stem cells; wherein the stem cell mixture contains less than about 0.5 EU/cc endotoxin; and wherein the stem cell mixture is at least about 95% viable as determined by flow cytometry.

In other embodiments, the invention is directed to methods for transferring autologous fat for cosmetic anatomical improvement, comprising the steps of: isolating stem cells from adipose tissue at a first anatomical location of a patient in need of cosmetic anatomical improvement; providing a stem cell composition derived from said isolated stem cells; and reimplanting an effective amount of a stem cell composition at a second anatomical location of said patient, wherein said second location requires said cosmetic anatomical improvement; and wherein said stem cell composition comprises: a population of stem cells obtained from mammalian adipose tissue; said population of stem cells comprising: endothelial progenitor cells, red blood cells, fibroblasts, white blood cells, hematopoietic stem cells and mesenchymal stem cells; wherein the stem cell mixture contains less than about 0.5 EU/cc endotoxin; and wherein the stem cell mixture is at least about 95% viable as determined by flow cytometry.

Cosmetic anatomical needs amenable to improvement include but are not limited to autologous fat transfer including for example, breast, facial, gluteal or other anatomical enhancement, or any combination thereof).

In yet other embodiments, the invention is directed to methods of rejuventating or improving the appearance of skin in a mammal, comprising the steps of: isolating stem cells from adipose tissue in a patient in need of skin appearance improvement or rejuvenation; providing a stem cell composition derived from said isolated stem cells; and reimplanting into said patient an effective amount of said stem cell composition into an epidermal anatomical region of said patient, wherein said region requires skin appearance improvement or rejuvenation; and wherein said stem cell composition comprises: a population of stem cells obtained from mammalian adipose tissue; said population of stem cells comprising: endothelial progenitor cells, red blood cells, fibroblasts, white blood cells, hematopoietic stem cells and mesenchymal stem cells; wherein the stem cell mixture contains less than about 0.5 EU/cc endotoxin; and wherein the stem cell mixture is at least about 95% viable as determined by flow cytometry.

Skin rejuvenations or improvements in appearance may include but are not limited to scar revisions, wound healing post cosmetic treatments, alopecia, and/or skin rejuvenation and combinations thereof.

In still other embodiments, the invention is directed to methods for modifying the shape of a mammalian anatomical region comprising the steps of: isolating stem cells from adipose tissue of a patient in need of an anatomical region shape modification; providing a stem cell composition derived from said isolated stem cells; and reimplanting into said patient an effective amount of a stem cell composition into said anatomical region of said patient, wherein said region is in need of said shape modification; and wherein said stem cell composition comprises: a population of stem cells obtained from mammalian adipose tissue; said population of stem cells comprising: endothelial progenitor cells, red blood cells, fibroblasts, white blood cells, hematopoietic stem cells and mesenchymal stem cells; wherein the stem cell mixture contains less than about 0.5 EU/cc endotoxin; and wherein the stem cell mixture is at least about 95% viable as determined by flow cytometry.

### BRIEF DESCRIPTION OF THE DRAWINGS

FIG. 1 illustrates a hemocytometer grid within a counting chamber.

FIG. 2 illustrates a representative square in a grid of single chamber of a hemocytometer.

FIG. 3 illustrates a typical hemocytometer slide as depicted from above and from a side on perspective.

### DETAILED DESCRIPTION OF ILLUSTRATIVE EMBODIMENTS

As employed above and throughout the disclosure, the following terms, unless otherwise indicated, shall be understood to have the following meanings.

As used herein, the term “Wunsch unit”, in reference to collagenase PZ activity, refers to an amount of the collagenase that can catalyze the hydrolysis of 1 μmol 4-phenyl-azobenzyloxycarbonyl-L-prolyl-L-leucyl-glycyl-L-prolyl-D-arginine per minute at 24° C., at pH 7.1.

As used herein, the term “stem cell” refers to an adult undifferentiated cell that can produce itself and a further differentiated progeny cell.

As used herein, the term “adipose tissue” refers to a diffuse organ of primary metabolic importance made-up of white fat, yellow fat or brown fat. The adipose tissue has adipocytes and stroma. Adipose tissue is found throughout the body of an animal. For example, in mammals, adipose tissue is present in the omentum, bone marrow, subcutaneous space and surrounding most organs.

As used herein “isolated” refers to a substance, for example an adipose-derived stem cell mixture, that is separated from contaminants (i.e. substances that differ from those components of a stem cell mixture as herein described.

As used herein, the term “effective amount” refers to an amount of stem cells or composition thereof as described herein that may be therapeutically effective to ameliorate, or treat the symptoms of particular disease, disorder, or side effect that is amenable to treatment with stem cells. Medical conditions amenable to treatment include but are not limited to osteoarthritis, Chronic Obstructive Pulmonary Disease (COPD), Erectile Dysfunction, Critical Limb Ischemia, Diabetes Mellitus, Type II Diabetes, Myocardial Infarction, Alzheimer's Disease, Autism, Cerebral Palsy, Congestive Heart Failure, Diffuse Brain Lesions, Frailty Syndrome, Multiple Sclerosis, Parkinson's Disease, Rheumatoid Arthritis, Renal Failure, Lupus, Stroke, Liver Failure, Ischemic Col iris, Spinal Cord Injury, degenerative disc disease, facet joint syndrome, sacroiliac joint dysfunction, ischemic congestive heart failure, Amyotrophic Lateral Sclerosis (ALS), and any combination thereof.

As used herein, the expressions “in combination with,” “combination therapy,” and “combination products” refer, in certain embodiments, to the administration to a patient of plasma rich fraction and stems cells or stem cell vascular fraction. When administered in combination, each component may be administered at the same time or sequentially in any order at different points in time. Thus, each component may be administered separately but sufficiently closely in time so as to provide the desired therapeutic effect.

As used herein, the term “dosage unit” refers to physically discrete units suited as unitary dosages for the particular individual to be treated. Each unit may contain a predetermined quantity of active stem cells or compositions thereof calculated to produce the desired therapeutic effect(s) or cosmetic effects. The specification for the dosage unit forms of the invention may be dictated by (a) the unique characteristics of the active stem cells, compositions thereof or combinations with other active agent(s) and the particular therapeutic effect(s) or cosmetic effects to be achieved, and (b) the limitations inherent in the art of compounding (or implanting of) such active stem cells or compositions thereof.

As used herein, the term “patient” refers to mammals, preferably humans.

When any variable occurs more than one time in any constituent, method, or, its definition in each occurrence is independent of its definition at every other occurrence. Combinations of substituents and/or variables are permissible only if such combinations result in viable products or compositions.

It is believed the biological descriptors and names used herein correctly and accurately reflect the underlying ingredients and/or biological entities. However, the nature and value of the present invention does not depend upon the theoretical correctness of these descriptors or names, in whole or in part. Thus it is understood that the descriptors and names used herein, as well as the biological names attributed to the correspondingly indicated components ingredients and/or biological entities, are not intended to limit the invention in any way.

In certain preferred embodiments, the stem cell compositions may further include platelet rich plasma. In other preferred embodiments, the methods of the present invention may incorporate the use of platelet rich plasma alone, in conjunction with, or in combination with any of the stem cell compositions of the present invention as herein described.

The present invention provides adipose-derived stem cells (ADSCs), their compositions, enzyme compositions useful in their manufacture, and methods for obtaining them from a mesodermal origin (e.g., adipose tissue) and using them.

The adipose derived stem cells, stem cell mixtures and/or compositions of the invention have the capacity to differentiate into mesodermal tissues, such as mature adipose tissue, bone, various tissues of the heart (e.g., pericardium, epicardium, epimyocardium, myocardium, pericardium, valve tissue, etc.), dermal connective tissue, hemangial tissues (e.g., corpuscles, endocardium, vascular epithelium, etc.), hematopeotic tissue, muscle tissues (including skeletal muscles, cardiac muscles, smooth muscles, etc.), urogenital tissues (e.g., kidney, pronephros, meta-and meso-nephric ducts, metanephric diverticulum, ureters, renal pelvis, collecting tubules, epithelium of the female reproductive structures (particularly the oviducts, uterus, and vagina), mesodermal glandular tissues (e.g., adrenal cortex tissues), and stromal tissues (e.g., bone marrow). Of course, inasmuch as the ADSC can retain potential to develop into a mature cell, it also can realize its developmental phenotypic potential by differentiating into an appropriate precursor cell (e.g., a preadipocyte, a premyocyte, a preosteocyte, etc.).

The inventive ADSCs are useful for, inter alia, tissue engineering, wound repair, in vivo and ex vivo tissue regeneration, tissue transplantation, and other methods that require cells that can differentiate into a variety of phenotypes and genotypes, or can support other cell types in vivo or in vitro.

One aspect of the invention pertains to an adipose-derived stromal vascular fraction that contains adipose-derived stem cells (ADSCs) of the invention. Typically, the stem cell mixtures of the present invention include endothelial progenitor cells, fibroblasts, pre-adipocytes, pericytes, macrophages, red blood cells, white blood cells, hemapoietic stem cells and mesenchymal stem cells. Alternately, the adipose-derived stem cell mixture is substantially free of one or more other cell types (e.g., adipocytes, red blood cells, and other stromal cells, etc.) and extracellular matrix material. Methods for its further purification to be substantially free of one or more other cell types may be adapted from methods known to those of ordinary skill in the art, for example, as disclosed in U.S. Pat. No. 7,470,537, hereby incorporated by reference herein in its entirety. The adipose-derived stromal vascular fraction is obtained from adipose tissue of a mammal. The preferred embodiment includes an adipose-derived stromal vascular fraction obtained from adipose tissue of a higher primate (e.g., a baboon or ape). The most preferred adipose-derived stromal vascular fraction is obtained from human adipose tissue, using the methods described herein.

The adipose-derived stromal vascular fractions of the invention are isolated from adipose tissue. The adipose tissue can be obtained from an animal by any suitable method. A first step in any such method requires the isolation of the adipose tissue from the source animal. The animal can be alive or dead, so long as adipose stromal cells within the animal are viable. Typically, human adipose tissue is obtained from a living donor, using well-recognized protocols such as surgical or suction lipectomy. The preferred method to obtain human adipose tissue is by excision or liposuction procedures well known in the art. Preferably, the inventive adipose-derived stromal vascular fractions are isolated from a liposuction aspirate (LPA). The adipose-derived stromal vascular fraction s of the invention are present in the initially excised or extracted adipose tissue, regardless of the method by which the adipose tissue is obtained.

However obtained, the adipose tissue is processed to separate the adipose-derived stromal vascular fraction(s) of the invention from the remainder of the adipose tissue. The adipose-derived stromal vascular fraction that contains the stem cells is usually obtained by washing the obtained adipose tissue with a physiologically-compatible solution, such as a cell wash solution. The washing step typically comprises a rinsing the adipose tissue with cell wash, agitating the tissue, and allowing the tissue to settle. In addition to washing, the adipose tissue is digested by enzyme degradation and neutralization. Preferably the enzyme degradation involves a blend of enzymes, preferably a proteolytic enzyme capable of digesting adipose tissue in combination with an enzyme that catalyzes the hydrolytic cleavage of phosphodiester linkages in DNA. The enzymes can contact the adipose tissue separately or in an admixture at substantially the same time, preferably they are added to the adipose tissue together. In conjunction with the enzymatic treatment, other dissociation methods may be used to further enhance digestion, such as mechanical agitation, sonic energy, or thermal energy. Once the adipose tissue has been sufficiently digested, the adipose mixture is allowed to settle, preferably encouraged to settle by using settling methods known to one of ordinary skill in the art. In one such embodiment, centrifugation is employed to assist in the settling of the layers. Typically, four layers form after the washing, digestion, and settling steps (including centriguation). The top layer is a free lipid or oil layer. The second layer contains adipocytes. The third layer contains media, and the bottom layer forms a pellet which includes the adipose-derived stromal vascular fraction containing the stem cells of interest.

The adipose-derived stromal vascular fraction may contain erythrocytes. In certain methods the erythrocytes are lysed and removed. Methods for lysis and removed erythrocytes are well known in the art (e.g., incubation in hypotonic medium). If the erythrocytes are removed, then the erythrocytes -free fraction contains the adipose-derived stromal vascular fraction and the stem cells of interest. However, the erythrocytes are not required to be removed from the adipose-derived stromal vascular fraction. In certain embodiments of the compositions or kits, the erythrocytes are removed. In alternative embodiments, the erythrocytes are not required to be removed. Similarly, in certain methods herein disclosed, the erythrocytes are removed, while in other alternative embodiments, they are not.

The pellet may be re-suspended and/or washed (in a re-suspension buffer), centrifuged, and re-suspended one or more successive times to achieve greater purity of the ADSCs. The adipose-derived stromal vascular fraction of the invention may be a heterogeneous population of cells which include the stem cells of the invention and adipocytes. The cells of the washed and re-suspended pellet are ready for plating if desired, but may be used directly in the methods of their use herein described.

Accordingly, in one embodiment, the present invention provides stem cell compositions comprising: a population of stem cells obtained from mammalian adipose tissue; said population of stem cells comprising: endothelial progenitor cells, red blood cells, fibroblasts, white blood cells, hematopoietic stem cells and mesenchymal stem cells; wherein the stem cell mixture contains less than about 0.5 EU/cc endotoxin; and wherein the stem cell mixture is at least about 95% viable as determined by flow cytometry.

In certain embodiments, the stem cell compositions are derived from human adipose tissue, preferably wherein the tissue donor and cell recipient are one and the same.

In other embodiments, the stem cells comprise endothelial progenitor cells, red blood cells, fibroblasts, white blood cells, hematopoietic stem cells and/or mesenchymal stem cells, and any combination thereof.

In yet other embodiments, the stem cell mixture contains less than about 2 EU/cc endotoxin, preferably less than about 1.5, more preferably less than about 1 with less than about 0.5 EU/cc endotoxin being even more preferred. In other embodiments, the stem cell mixture contains only a de minimis quantity of endotoxin.

In still other embodiments, the stem cell mixture is at least about 75% viable, preferably about least about 80%, more preferably at least about 85%, still more preferably at least about 90% with at least about 95% viability as determined by flow cytometry being even more preferred. In certain other embodiments, the stem cell mixture is at least about 95%, preferably at least about 96, more preferably at least about 97, still more preferably at least about 98% viable as determined by flow cytometry. In other preferred embodiments, the stem cell mixture contains less than about 0.5 EU/cc endotoxin and is at least about 95% viable as determined by flow cytometry.

In some embodiments, the ratio of hematopoietic stem cells to mesenchymal stem cells present in the stem cell composition is in the range of from about 1:4 to about 4:1 as determined by flow cytometry, preferably from about 1:3 to about 3:1, more preferably from about 2:3 to about 3:2. In certain alternative embodiments, the ratio of hematopoietic stem cells to mesenchymal stem cells present in the stem cell composition is in the range of from about 1:4 to about 1:1.

In certain embodiments, the stem cell composition contains from about 15 to about 30%, preferably from about 20 to about 25% hematopoietic stem cells, and from about 25 to about 40%, preferably from about 30 to about 35% mesenchymal stem cells, based on the total number of cells in the composition.

In certain other embodiments, the stem cell compositions further comprise at least one of: preadipocytes, pericytes and macrophages, or any combination thereof.

In another aspect of the present invention, enzyme compositions are provided that comprise: a proteolytic enzyme capable of digesting adipose tissue; and an enzyme that catalyzes the hydrolytic cleavage of phosphodiester linkages in DNA.

The enzyme compositions are generally useful for the digestion of adipose tissue, including human adipose tissue, and find additional beneficial use in processes employed for the isolation and/or recovery of stem cells from the digested adipose tissue. The enzyme composition used for the dissociation of said adipose tissue typically contains from about 0.25 to about 0.75 Wunsch units, or from about 250 to about 750 Collagen Digestion Units of activity per cubic centimeter of adipose tissue. In other embodiments, the enzyme compositions contain from about 7 to about 15 Kunitz units of DNase I per cubic centimeter of adipose tissue; more preferably, they contain from about 0.25 to about 0.75 Wunsch units or from about 250 to about 750 Collagen Digestion Units and from about 7 to about 15 Kunitz units of DNase I per cubic centimeter of adipose tissue.

In some preferred embodiments, the proteolytic enzyme capable of digesting adipose tissue comprises a collagenase, more preferably a collagenase derived from Clostridium histolyticum. The collagenase, preferably derived from Clostridium histolyticum, typically has a molecular weight in the range of rom about 70 to about 120 kDa and contains about 85% protein by Lowery assay. While the source of the enzymes is not critical to the performance of the enzynme blend in methods of isolation described herein, exemplary collagenase having an activity of at least about 0.18 Wunsch units/mg collagenase lyophilisate can be obtained from Serva Electrophoresis GmbH, Heidelberg, Germany. In other preferred embodiments, the enzyme that catalyzes the hydrolytic cleavage of phosphodiester linkages in DNA comprises a DNAse. Exemplary DNAase that catalyzes the hydrolytic cleavage of phosphodiester linkages in DNA e can be obtained from Roche Diagnostics GmbH, Roche Applied Sciened, 68298 Mannheim, Germany. More preferably, the enzyme composition comprises a collagenase and a DNAse.

Other aspects of the invention are directed to methods for isolating stem cells from mammalian adipose tissue comprising the steps of: contacting mammalian adipose tissue with an enzyme composition as disclosed herein for a time and under conditions effective to digest the adipose tissue; and isolating the cells from the enzyme digested adipose tissue; wherein the adipose tissue was extracted from a mammal.

In some preferred embodiments, the methods for isolating stem cells further comprise a mammalian adipose tissue extraction process comprising the steps of: inserting a cannula into a subcutaneous space containing adipocytes in said mammal; wherein the cannula is attached to a syringe; aspirating said subcutaneous space for a time and under conditions effective to transfer said adipocytes optionally containing an amount of ruptured adipocytes or blood components from the subcutaneous space via cannula to the syringe; and rinsing the adipocytes for a time and under conditions effective to reduce the amount of ruptured adipocytes or blood components contained in the transferred adipocytes to provide the extracted mammalian adipose tissue for isolating the stem cells.

In some other preferred embodiments, the adipocytes contain an amount of ruptured adipocytes or blood components; more preferably, in those instances where ruptured adipocytes or blood components are present in the LPA, their levels may be reduced by rinsing the adipocytes, for example with a cell wash solution or other neutral medium.

In yet other embodiments, the invention is directed to methods for treating a medical condition amenable to treatment with stem cells.

In some preferred embodiments, the present methods are directed to treating one or more conditions selected from the group consisting of osteoarthritis, Chronic Obstructive Pulmonary Disease (COPD), Erectile Dysfunction, Critical Limb Ischemia, Diabetes Mellitus, Type II Diabetes, Myocardial Infarction, Alzheimer's Disease, Autism, Cerebral Palsy, Congestive Heart Failure, Diffuse Brain Lesions, Frailty Syndrome, Multiple Sclerosis, Parkinson's Disease, Rheumatoid Arthritis, Renal Failure, Lupus, Stroke, Liver Failure, Ischemic Col iris, Spinal Cord Injury, or Amyotrophic Lateral Sclerosis (ALS), and any combination thereof.

In certain embodiments of methods for treating a medical condition amenable to treatment with stem cells in a patient in need thereof, the methods comprise the step of: isolating stem cells from adipose tissue in a patient in need of such treatment; providing a stem cell composition derived from the isolated stem cells; and reimplanting an effective amount of a stem cell composition of the present invention into an anatomical region of-said patient, wherein the region is affected with the medical condition being treated.

In yet other embodiments, the invention is directed to methods for transferring autologous fat for cosmetic anatomical improvement. In certain preferred embodiments the cosmetic anatomical needs amenable to improvement include but are not limited to autologous fat transfer for enhancements of, for example, breast, facial, gluteal or other anatomical tissue, or any combination thereof.

In certain embodiments of methods for transferring autologous fat for cosmetic anatomical improvement amenable to implantation of stem cells in a patient in need thereof, the methods comprise the step of: the invention is directed to methods for transferring autologous fat for cosmetic anatomical improvement, comprising the steps of: isolating stem cells from adipose tissue at a first anatomical location of a patient in need of cosmetic anatomical improvement; providing a stem cell composition according to the present invention as derived from said isolated stem cells; and reimplanting an effective amount of a stem cell composition at a second anatomical location of said patient, wherein said second location requires said cosmetic anatomical improvement.

In yet other embodiments, the invention is directed to methods for rejuventating skin or improving the appearance of skin in a mammal. Skin rejuvenations or improvements in appearance may include but are not limited to scar revisions, wound healing post cosmetic treatments, alopecia, and/or skin rejuvenation and combinations thereof.

In certain embodiments of methods for rejuventating skin or improving the appearance of skin in a mammal amenable to implantation of stem cells in a patient in need thereof, the methods comprise the steps of: isolating stem cells from adipose tissue in a patient in need thereof; providing a stem cell composition derived from said isolated stem cells; and reimplanting into an epidermal anatomical region of said patient's body an effective amount of a stem cell composition according to the present invention.

In still other embodiments, the invention is directed to methods for modifying the shape of a mammalian anatomical region wherein the region is amenable to shape modification by the implantation of stem cells. The modification may include the addition of stem cells to enlarge the appearance of the region or increase the cell population in a given region. Alternatively, the modification may involve the removal of certain tissue to enhance overall appearance, such removal being accomplished by any of the methods known to the ordinarily skilled artisan. Once tissue is removed from the area being modified, it may be beneficial to implant a stem cell composition as a way of “fine-tuning” the modification. Any of the various regions of the anatomy may be considered for an enlarging or reducing (or combination thereof) modification as would be recognized by one or ordinary skill in the art.

In certain embodiments of methods for modifying the shape of a mammalian anatomical region wherein the region is amenable to shape modification by the implantation of stem cells, the methods comprise the steps of: isolating stem cells from adipose tissue of a patient in need of an anatomical region shape modification; providing a stem cell composition derived from said isolated stem cells; and reimplanting into said patient at said mammalian anatomical region in need of said shape modification an effective amount of a stem cell composition according to the present invention.

The stem cells, or compositions thereof, employed in the methods of the present invention may be prepared in a number of ways well known to those skilled in the art. The stem cells or compositions thereof can be prepared, for example, by the methods described herein, or variations thereon as appreciated by the skilled artisan. All processes disclosed in association with the present invention are contemplated to be practiced on any scale, including milligram, gram, multigram, kilogram, multikilogram or commercial industrial scale. In certain embodiments, they are preferably practiced on a scale as dictated by the needs of the particular patient in relationship to the availability of the patient's adipose tissue. Generally, speaking it is useful to prepare only enough stem cells so that a single treatment is necessary to implant them reasonably as soon as they are isolated, to ensure maximum viability of the cells to be implanted.

As noted above, certain embodiments of the present invention may involve, inter alia, platelet rich plasma. The platelet rich plasma (PRP) employed in any of the compositions, kits and/or methods herein described, may be prepared by any means understood to one of ordinary skill in the art. An exemplary method for preparing the PRP is provided for illustrational purposes in the experimental section of the present application, and not meant to be limiting. PRP products preferably prepared by methods disclosed herein may be classified as pure platelet rich plasma (P-PRP) according to the classification system proposed by Dohan Ehrenfest et al. Dohan Ehrenfest et al., Trends Biotechnol. 2009; 27: 158-167. These PRP harvesting and isolation protocols provide from about 2 to about 6 cc, preferably from about 3 to about 5 cc of PRP from a 30 cc venous blood draw. The platelet count meets the working definition of PRP, which is 10 lakh/ml in 5 ml of PRP according to Marx. Marx, R. E., Implant Dent. 2001; 10: 225-228.

Other optional conventional stem cell components, and optional compounds for enhancing the potency or viability of the stem cell components that may be employed in the methods and compositions of the present invention, in addition to those exemplified above, would be readily apparent to one of ordinary skill in the art, once armed with the teachings of the present disclosure.

Although the stem cells of the present invention may be reimplanted as highly purified cells, it is preferable to present the viable cells as a pharmaceutical composition. The invention thus further provides a pharmaceutical composition comprising an effective amount of one or more types of stem cells of the invention, preferably one or more types of cells, as described herein, together with one or more pharmaceutically acceptable carriers therefore and, optionally, other therapeutic and/or prophylactic ingredients, such as for example, platelet rich plasma. The carrier(s) must be acceptable in the sense of being compatible with the other ingredients of the composition and not deleterious to the recipient thereof.

The compositions of the invention may be administered in an effective amount by any of the conventional techniques well-established in the medical field. The compositions employed in the methods of the present invention including, for example, platelet rich plasma and the stem cell compositions of the invention, preferably those comprising endothelial progenitor cells, red blood cells, fibroblasts, white blood cells, hematopoietic stem cells and mesenchymal stem cells, as described herein, may be administered by any means that results in the contact or inplantation of the active stem cell composition(s) with the relevant site or site(s) of action in the body of a patient. The compounds may be administered by any conventional means available for use in conjunction with stem cell compositions, either as individual therapeutic agents or in a combination of therapeutic agents. For example, they may be administered as the sole active agents in a pharmaceutical composition, or they can be used in combination with other therapeutically active ingredients.

Stem cells and compositions thereof of the present invention, preferably those comprising endothelial progenitor cells, red blood cells, fibroblasts, white blood cells, hematopoietic stem cells and mesenchymal stem cells, as described herein, may be administered to a mammalian host in a variety of forms adapted to the chosen route of administration, e.g., typically by injection. The term injection encompasses intravenous (IV), intramuscular (IM), and/or subcutaneous (SC) administration.

The pharmaceutical forms suitable for injectable use include, for example, sterile aqueous solutions or dispersions. In all cases, the form is preferably sterile and fluid to provide easy syringability. It is preferably stable under the conditions of manufacture and storage and is preferably preserved against the contaminating action of microorganisms such as bacteria and fungi. It is preferably employed within a reasonable time after it is prepared, more preferably within an hour or two, or less of its preparation, although its viability may be maintained or extended by practices known to those of skill in the art. The carrier may be a solvent or dispersion medium containing, for example, water, or PRP or suitable mixtures thereof. In some cases, it may be preferable to include isotonic agents, for example, sugars, or sodium chloride.

Sterile injectable solutions may be prepared by incorporating the active stem cells in the required amounts, in the appropriate solvent, with various of the other ingredients enumerated above, as required, followed by filtered sterilization. Generally, dispersions may be prepared by incorporating the sterilized active ingredient into a sterile vehicle which contains the basic dispersion medium and the required other ingredients from those enumerated above.

The therapeutic compounds of this invention may be administered to a patient alone or in combination with a pharmaceutically acceptable carrier. The relative proportions of active ingredient and carrier may be determined, for example, by the solubility and biologically viable nature of the compounds, chosen route of administration, and standard pharmaceutical practice.

The dosage of the stem cell compositions of the present invention that will be most suitable for prophylaxis or treatment will vary with the form of administration, the particular medical condition or cosmetic improvement chosen and the physiological characteristics of the particular patient under treatment. Generally, small dosages may be used initially and, if necessary, increased by small increments until the desired effect under the circumstances is reached. Alternatively, follow up dosages may be administered to a patient over time to effect or maintain or enhance the desired improvement. Although the proper dosage of the compositions of this invention will be readily ascertainable by one skilled in the art, once armed with the present disclosure, by way of general guidance, for example, typically a unit dosage of the composition of the invention as described herein, is typically determined based on condition treated and mode of delivery (i.e., intravenous, inhalation, intranasal, intra-articularly) as well as by the amount of adipose tissue collected. A typical standard dose is delivered from either 60 cc or 120 cc of supernatant lipoaspirate. Based on validation studies, 60 cc of supernatant lipoaspirate yields in the range of from about 4 million to about 8 million, preferably from about 5 million to about 7 million, more preferably from about 5.5 million to about 6.6 million mesenchymal stem cells utilizing the proposed invention and method. As noted herein, the number of doses required is a function of the condition being treated and the manner of the dosage's administration. For example, a dosage derived from 60 cc of supernatant lipoaspirate may be appropriate for treating certain conditions such as osteoarthritis or wound repair, preferably used in combination with PRP and/or when administration is carried out intra-articularly. When employing dosages for certain treating some conditions, it is preferable to administer a double dosage of the mesenchymal stem cells (i.e., a stem cell dosage typically isolated from about 120 cc of supernatant lipoaspirate when employing the isolation techniques disclosed herein).

In some embodiments of the present invention the mode of delivery is not critical insofar as it is capable of delivering the stem cells and/or PRP to the desired location of treatment and/or does not render the stem cells and/or PRP ineffective for treatment of the particular medical condition or cosmetic improvement chosen. However, for some conditions, preferred methods may enhance or facilitate treatment. For example, in certain preferred embodiments, intra-articular delivery is a preferred delivery method for the treatment of osteoarthritis. In other preferred embodiments, intra-articular delivery is a preferred delivery method for the treatment of osteoarthritis, facet joint dysfunction or sacroiliac joint dysfunction. In yet other preferred embodiments, intra-articular delivery and/or intravenous delivery are preferred delivery methods for the treatment of rheumatoid arthritis. In some other preferred embodiments, intravenous delivery is a preferred delivery method for the treatment of Type II diabetes, Frailty syndrome, or ischemic congestive heart failure. In still other preferred embodiments, intranasal delivery and/or intravenous delivery are preferred delivery methods for the treatment of Multiple sclerosis, Parkinson's Disease, or Alzheimer's Disease. In other preferred embodiments, intramuscular delivery is a preferred delivery method for the treatment of critical limb ischemia. In some other preferred embodiments, inhalation delivery and/or intravenous delivery are preferred delivery methods for the treatment of COPD. In certain other preferred embodiments, direct injection into the corpus cavernosum is the preferred delivery method for the treatment of erectile dysfunction. In other preferred embodiments, intradiscal delivery is the preferred delivery method for the treatment of degenerative disc disease.

The combination products of this invention, such as pharmaceutical compositions comprising platelet rich plasma in combination with one or more stem cell compositions of the invention, as described herein, or one or more pharmaceutically acceptable salts thereof, may be in any dosage form, such as those described herein, and can also be administered in various ways, as described herein. In a preferred embodiment, the combination products of the invention comprise the stem cell compositions of the present invention and PRP, each preferably prepared by the methods disclosed herein. In some preferred embodiments, the combination products of the present invention consist essentially of the stem cell compositions of the present invention and PRP, more preferably consist of the stem cell compositions of the present invention and PRP. By way of general guidance, when the stem cell composition and PRP are formulated together, a typical stem cell composition isolated from about 60 cc of supernatant lipoaspirate may be suspended in the PRP (from about 3 to about 5 cc) that may be obtained from a 30 cc venous blood draw as disclosed herein to provide a single dosage form (that is, combined together, for example, in one injection, preferably an intra-articular injection).

When the combination products are not formulated together in a single dosage form, the platelet rich plasma and the stem cell composition of the invention, may be administered at the same time (that is, together), or in any order. When not administered at the same time, preferably the administration of PRP and the stem cell compound of the invention occur less than about one hour apart, more preferably less than about 30 minutes apart, even more preferably less than about 15 minutes apart, and still more preferably less than about 5 minutes apart. Preferably, administration of the combination products of the invention is via injection, although other routes of administration, as described above, such as for example, intrasanal, inhalation or intravenous administration, are contemplated to be within the scope of the present invention. Although it is preferable that the stem cell composition and the PRP are both administered in the same fashion (that is, for example, both by subcutaneous injection), if desired, they may each be administered in different fashions (that is, for example, one component of the combination product may be administered subcutaneously, and another component may be administered by intra-articular injection. The dosage of the combination products of the invention may vary depending upon various factors such as its mode and route of administration, the age, health and weight of the recipient, the nature and extent of the symptoms, the kind of concurrent treatment, the frequency of treatment, and the effect desired.

In any of the methods of treatment disclosed herein, it may be further advantageous to administer PRP at a time after the administration of the stem cell composition or stem cell/PRP composition, for example to enhance the therapeutic benefit of the condition's treatment and to boost or further stimulate response from the implanted or otherwise administered stem cells in a patient.

Although the proper dosage of the combination products of this invention will be readily ascertainable by one skilled in the art, once armed with the present disclosure, by way of general guidance, where platelet rich plasma is combined with a stem cell composition of the invention, as described herein, for example, typically a unit dosage may range from about 4 million to about 8 million, preferably from about 5 million to about 7 million, more preferably from about 5.5 million to about 6.6 million mesenchymal stem cells per 60 cc of supernatant lipoaspirate harvested from the patient. With regard to a typical dosage form of this type of combination product of stem cells and PRP, the PRP from about a 30 cc venous draw providing from about 3 to about 5 cc of PRP is suitable in which to suspend the stem cells derived from the about 60 cc of supernatant lipoaspirate. Larger dosages may be provided to a patient by isolating a greater number of stem cells (starting with a larger volume of aspirate) and isolating a greater amount of PRP via a larger volume venous draw. For at least the reason of providing consistency in administration, it is preferable to maintain the ratio of stem cells to PRP as hereindisclosed.

A typical standard dose is delivered from either 60 cc or 120 cc of supernatant lipoaspirate. Based on validation studies, 60 cc of supernatant lipoaspirate yields in the range of from about 4 million to about 8 million, preferably from about 5 million to about 7 million, more preferably from about 5.5 million to about 6.6 million mesenchymal stem cells utilizing the proposed invention and method. As noted herein, the number of doses required is a function of the condition being treated and the manner of the dosage's administration.

Pharmaceutical kits useful in, for example, methods for isolation of stem cells from adipose tissue, which comprise a catalytically effective amount of an enzyme composition of the present invention, in one or more sterile containers, are also within the ambit of the present invention. Sterilization of the container may be carried out using conventional sterilization methodology well known to those skilled in the art. The kits may also contain one or more of the following components, or any combination thereof: a syringe, preferably luer lock type, 100 μm nylon cell strainer, 0.2/100 μm syringe filter, cell wash solution (for example, Hank's Balanced Salt Solution, from Sigma-Aldrich, St. Louis, Mo.), a cell extractor (vessel dilator 12 F), a 50 mL conical tube, and a 250 ml cell wash bag. In certain embodiments of the kits of the present invention, a plurality of one or more of the components may be included, for example, in amounts as needed to complete a stem cell isolation process as described herein. Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit.

It will be further appreciated that the amount of the compound, or an active salt or derivative thereof, required for use in treatment will vary not only with the particular stem cell composition prepared but also with the route of administration, the nature of the condition being treated and the age and condition of the patient and will be ultimately at the discretion of the attendant physician or clinician.

The desired dose of composition may conveniently be presented in a single dose or as divided doses administered at appropriate intervals, for example, as two, three, four or more sub-doses per visit. The sub-dose itself may be further divided, e.g., into a number of discrete loosely spaced administrations; such as multiple injections from a cannula.

The enzyme compositions of the present invention may be used in methods to isolate stem cells from mammalian adipose tissue. Such isolation may be accomplished by contacting adipose tissue, for example, lipoaspirate, with an effective amount of a proteolytic enzyme capable of digesting adipose tissue and an effective amount of an enzyme that catalyzes the hydrolytic cleavage of phosphodiester linkages in DNA. Preferably, the contacting step conducted in an aqueous medium, preferably at physiologically relevant ionic strength, pH, and the like.

In preferred embodiments of the isolation methods of the invention, the enzymes digest the adipose tissue and catalyze the hydrolytic cleavage of phosphodiester linkages in DNA.

The stem cell compositions of the present invention may be used in methods to treat or ameliorate certain medical conditions amenable to treatment with stem cells, or any combinations or subcombinations of those conditions. Furthermore, the compositions of the invention may be used as to provide patients with cosmetic anatomical improvement, skin rejuvenation, anatomical shape modification, or skin appearance improvement s that are ameliorated by stem cells or in any treatment wherein such cell regeneration is desired.

Such symptoms, conditions or diseases include the osteoarthritis, Chronic Obstructive Pulmonary Disease (COPD), Erectile Dysfunction, Critical Limb Ischemia, Diabetes Mellitus, Type II Diabetes, Myocardial Infarction, Alzheimer's Disease, Autism, Cerebral Palsy, Congestive Heart Failure, Diffuse Brain Lesions, Frailty Syndrome, Multiple Sclerosis, Parkinson's Disease, Rheumatoid Arthritis, Renal Failure, Lupus, Stroke, Liver Failure, Ischemic Col iris, Spinal Cord Injury, or Amyotrophic Lateral Sclerosis (ALS), and any combination thereof.

Certain aspects of the invention provide the following non-limiting embodiments:

### Embodiment 1

A stem cell composition comprising: a population of stem cells obtained from mammalian adipose tissue; said population of stem cells comprising: endothelial progenitor cells, red blood cells, fibroblasts, white blood cells, hematopoietic stem cells and mesenchymal stem cells; wherein the stem cell mixture contains less than about 0.5 EU/cc endotoxin; and wherein the stem cell mixture is at least about 95% viable as determined by flow cytometry.

### Embodiment 2

A stem cell composition according to Embodiment 1, wherein the stem cell mixture is at least about 97% viable as determined by flow cytometry.

### Embodiment 3

A stem cell composition according to Embodiment 1, wherein the stem cell mixture is at least about 98% viable as determined by flow cytometry.

### Embodiment 4

A stem cell composition according to any of Embodiments 1-3, wherein the ratio of hematopoietic stem cells to mesenchymal stem cells is in the range of from about 1:4 to about 4:1 as determined by flow cytometry.

### Embodiment 5

A stem cell composition according to any of Embodiments 1-3, wherein the ratio of hematopoietic stem cells to mesenchymal stem cells is in the range of from about 2:3 to about 3:2.

### Embodiment 6

A stem cell composition according to any of Embodiments 1-3, wherein the ratio of hematopoietic stem cells to mesenchymal stem cells is in the range of from about 1:4 to about 1:1.

### Embodiment 7

A stem cell composition according to Embodiment 4, wherein the composition contains from about 20 to about 25% hematopoietic stem cells and from about 30 to about 35% mesenchymal stem cells.

### Embodiment 8

A stem cell composition according to any of Embodiments 1-7, further comprising at least one of: preadipocytes, pericytes and macrophages.

### Embodiment 9

A stem cell composition according to any of Embodiments 1-8, wherein the mammalian adipose tissue is harvested from a human.

### Embodiment 10

An enzyme composition comprising: a proteolytic enzyme capable of digesting adipose tissue; and an enzyme that catalyzes the hydrolytic cleavage of phosphodiester linkages in DNA; wherein the enzyme composition used for the dissociation of said adipose tissue contains from about 0.25 to about 0.75 Wunsch units or from about 250 to about 750 Collagen Digestion Units and from about 7 to about 15 Kunitz units of DNase I per cubic centimeter of adipose tissue.

### Embodiment 11

An enzyme composition according to Embodiment 10, wherein the proteolytic enzyme capable of digesting adipose tissue is a collagenase and the enzyme that catalyzes the hydrolytic cleavage of phosphodiester linkages in DNA is a DNAse.

### Embodiment 12

An enzyme composition according to Embodiment 10, wherein the proteolytic enzyme capable of digesting adipose tissue is a collagenase derived from Clostridium histolyticum.

### Embodiment 13

A pharmaceutical kit for isolation of stem cells from mammalian adipose tissue comprising a container having an effective amount of an adipose digestion enzyme composition according to any of Embodiments 10-12.

### Embodiment 14

A kit according to Embodiment 13, further comprising conventional stem cell isolation kit components.

### Embodiment 15

A kit according to Embodiment 14, wherein said conventional stem cell isolation kit components include at least one of the following: a syringe, cell strainer, syringe filter, cell wash solution, cell extractor, conical tube, cell wash bag, and sterile container with lid.

### Embodiment 16

A kit according to any of Embodiments 13-15, further comprising instructions for isolating stem cells from mammalian adipose tissue.

### Embodiment 17

A method for isolating stem cells from mammalian adipose tissue comprising the steps of: contacting mammalian adipose tissue with an enzyme composition according to any of embodiments 10-12 for a time and under conditions effective to digest the adipose tissue; and isolating the cells from the enzyme digested adipose tissue; wherein the adipose tissue was extracted from a mammal.

### Embodiment 18

A method according to Embodiment 17 further comprising a mammalian adipose tissue extraction process comprising the steps of: inserting a cannula into a subcutaneous space containing adipocytes in said mammal; wherein the cannula is attached to a syringe; aspirating said subcutaneous space for a time and under conditions effective to transfer said adipocytes containing an amount of ruptured adipocytes or blood components from the subcutaneous space via cannula to the syringe; andrinsing the adipocytes for a time and under conditions effective to reduce the amount of ruptured adipocytes or blood components contained in the transferred adipocytes and to provide the extracted mammalian adipose tissue.

### Embodiment 19

A stem cell composition prepared by the process according to any of Embodiments 17-18.

### Embodiment 20

A method for treating a medical condition amenable to treatment with stem cells comprising the steps of: isolating stem cells from adipose tissue of a patient in need of treatment for a medical condition amenable to treatment with stem cells; providing a stem cell composition according to any of Embodiments 1-9 comprising said isolated stem cells; and reimplanting an effective amount of said stem cell composition into an anatomical region of-said patient, wherein said region is affected with said medical condition.

### Embodiment 21

A method for transferring autologous fat for cosmetic anatomical improvement, comprising the steps of: isolating stem cells from adipose tissue at a first anatomical location of a patient in need of cosmetic anatomical improvement; providing a stem cell composition according to any of Embodiments 1-9 comprising said isolated stem cells; and reimplanting an effective amount of said stem cell composition at a second anatomical location of said patient, wherein said second location requires said cosmetic anatomical improvement.

### Embodiment 22

A method for rejuvenating skin or improving the appearance of skin in a mammal, comprising the steps of: isolating stem cells from adipose tissue in a patient in need of skin appearance improvement or rejuvenation; providing a stem cell composition according to any of Embodiments 1-9 comprising said isolated stem cells; and reimplanting an effective amount of said stem cell composition into an epidermal anatomical region of said patient, wherein said region requires said skin appearance improvement or rejuvenation.

### Embodiment 23

A method for modifying the shape of a mammalian anatomical region comprising the steps of: isolating stem cells from adipose tissue of a patient in need of an anatomical region shape modification; providing a stem cell composition according to any of Embodiments 1-9 comprising said isolated stem cells; and reimplanting an effective amount of said stem cell composition into an anatomical region of said patient, wherein said region requires shape modification.

### Embodiment 24

An enzyme composition comprising: a proteolytic enzyme capable of digesting human adipose tissue; and an enzyme that catalyzes the hydrolytic cleavage of phosphodiester linkages in DNA; wherein said enzyme composition is sufficient to digest said human adipose tissue, and provide a stem cell composition containing at least about 2.0E+05 cells per cc LPA.

### Embodiment 25

The enzyme composition according to Embodiment 24, wherein the stem cell composition is at least about 95% viable as determined by flow cytometry.

### Embodiment 26

The enzyme composition according to any of Embodiments 24-25, wherein the stem cell composition contains from about 20 to about 25% hematopoietic stem cells and from about 30 to about 35% mesenchymal stem cells as determined by flow cytometry.

### Embodiment 27

A pharmaceutical composition comprising: a stem cell composition according to any of Embodiments 1-9; and platelet rich plasma.

### Embodiment 28

A pharmaceutical composition consisting of: a stem cell composition according to any of Embodiments 1-9; and platelet rich plasma.

### Methods of Preparation

Aspects of the present invention will now be illustrated by reference to the following specific, non-limiting examples. Those skilled in the art of stem cell isolation or purification may be aware of still other routes to the compositions, kits and methods of the present invention. The reagents and intermediates used herein are either commercially available or prepared according to standard literature procedures.

### Experimental Section

### I. Procedure for Isolation of Human Adipose Derived Stem Cells

This procedure describes the enzymatic digestion and isolation of adipose derived stem cells (ADSCs) from human adipose samples.

### Equipment—

HEPA filtered, laminar flow biosafety cabinet, shaker/incubator, 250 ml plastic beaker, 60 cc luer lock syringe, 20 cc luer lock syringe, 50 ml conical tube, 0.2 μm filter, 100 μm net well strainer, cell extractor, cell wash Solution, wash bag

Spray a plastic beaker, cell wash solution bottle, 60 cc syringe, and cell extractor with sterile alcohol and transfer to laminar flow biosafety cabinet. Obtain approximately 60 cc of adipose lipoaspirate from an adipose tissue donor, spray the syringe containing the aspirate with sterile alcohol, and transfer the syringe to the laminar flow biosafety cabinet. Place the lipoaspirate syringe in a syringe caddy, cap down, to allow fluid to settle to bottom. Remove excess fluid by gently pushing the excess fluid from the lipoaspirate syringe into the plastic beaker (waste). Open the small port of the cell wash bag, attach the syringe containing the remaining aspirate, and transfer the aspirate into the bag.

Using the previously sterilized (alcohol sprayed) 60 cc syringe and a cell extractor, attach the cell extractor to the 60 cc syringe and withdraw 60 ml of Cell Wash Solution from its container. Remove the cell extractor from the 60 cc syringe and place into the waste container, and add the cell wash solution into the cell wash bag. Attach the luer lock of the 60 cc lipoaspirate syringe to the cell wash bag and add the entire contents of the bag. Leave the luer lock connected to the bag. Agitate the tissue by inverting approximately 5 times, and allow the fat to separate for a period of 2 to 4 minutes with the luer lock and syringe pointing downward. Collect the fluid into the syringe being careful not to drain any fat. Flip bag and disconnect syringe. Drain fluid from the syringe into waste container.

### Enzymatic Digestion to Break Down Fat Tissue and Release Cells

Spray the following items (enzyme-blend-containing vial, 0.2 μm filter, 60 cc syringe, cell extractor, and 50 ml conical tube) with alcohol, and then aseptically transfer them to the laminar flow biosafety cabinet. Using a cell extractor and a 60 cc syringe, collect 50 ml Cell Wash Solution from the cell wash solution bottle. Add approximately 45 ml of Cell Wash Solution to a 50 ml conical tube. Open the vial containing the enzyme blend composition and empty the enzyme blend into the 50 ml conical tube containing 45 cc of cell wash solution. Add the remaining 5 cc of cell wash solution to the enzyme vial and transfer contents to the 50 ml conical tube. Using the 60 cc syringe and cell extractor, collect the 50 ml of suspended enzyme blend. Remove the cell extractor and attach the 0.2 μm filter to the 60 cc syringe. Transfer enzyme solution via syringe into the cell wash bag through the filter. Place the cell wash bag into the shaker/incubator and shake for 30 minutes at 37° C. and 225 rpm shaker setting. Alternatively, the cell wash bag may be shaken every ten minutes for thirty minutes, if using an incubator or dry block only. After thirty minutes, minutes shake the bag vigorously for approximately 30 seconds to break up remaining tissue.

Spray four 50 ml conical tubes with alcohol and transfer them to the laminar flow biosafety cabinet. Transfer the adipose tissue evenly into the four conical tubes using the large port of the cell bag Centrifuge the enzyme-digested adipose tissue at 2000 rpm and room temperature for 5 minutes. Do not use the brake to stop the centrifuge as this will adversely affect separation. After centrifugation, the following layers will be observed, in this order from top to bottom: oil, adipocytes, media, and stromal vascular fraction (SVF). The SVF will form a pellet at the bottom of the tube.

### Cell Isolation of the SVF

Spray a 20 cc syringe, a 50 ml conical tube and a cell extractor with alcohol and transfer them to the laminar flow biosafety cabinet. Using a 20 cc syringe and a cell extractor, transfer approximately 20 ml of Cell Wash Solution to the 50 ml conical tube. Using the 20 cc syringe and cell extractor from above, withdraw the SVF cell pellet at the bottom of the each of the centrifuged conical tubes. Transfer each of the pellets into a single 50 ml conical tube containing 20 ml of Cell Wash Solution and triturate the pellets to ensure that they are broken up completely. Centrifuge conical tube containing the SVF pellets at 2000 rpm and room temperature for 5 minutes. Do not use the brake to stop the centrifuge.

### Final Filtration and Re-Suspension to Remove Fibrous Tissue and Create Single Cell Suspension

Spray a 20 cc syringe and a cell extractor with alcohol and transfer to the Laminar Flow Biosafety Cabinet. Aspirate the supernatant from the centrifuged tube in the previous step using the 20 cc syringe and cell extractor. Spray a 20 cc syringe, a 50 ml conical tube, cell strainer and a cell extractor with alcohol and transfer to the Laminar Flow Biosafety Cabinet. Re-suspend the cells in 20 ml of Cell Wash Solution in a conical tube using a 20 cc syringe and a cell extractor. If applicable, obtain 20 μl for cell counts and viability using a hemocytometer. Transfer the sample to microscope and perform counts according to Cell Counting SOP. A 1:3 dilution is recommended as a starting point. Place a 100 μl strainer in the 50 ml conical tube and pass the entire cell suspension through the strainer. Use a steady flow from the pipet and a swirling motion to assist the digested tissue through the strainer. The solution may be thick and require time to filter. Centrifuge conical tube containing the strained cell suspension at 2000 rpm and room temperature for 5 minutes. Do not use the brake.

### Final Re-Suspension

Spray a 20 cc syringe and a cell extractor with alcohol and transfer to the laminar flow biosafety cabinet. Using the 20 cc syringe and cell extractor, carefully aspirate as much of the supernatant in the centrifuged conical tube as possible without aspirating the pelleted cells. Obtain a syringe containing final re-suspension buffer {approximately 1-6 mls) Attach the cell extractor to the syringe and re-suspend the cells in the final re-suspension solution. Draw the cells mixed in re-suspension solution back into the final re-suspension syringe. Remove the cell extractor and cap the syringe. Transfer to the operating room for immediate use.

### II. Procedure for Isolation of Human Platelet Rich Plasma

### Materials—

20 cc luer lock syringe, 3-5 cc syringe, 50 ml conical tube (2), cell extractor (2), blood collection tubing set, venous blood collection tubes (4)

Collect approximately 30-35 ml of peripheral blood using the blood collection tubing set and the 4 blood collection tubes. Thoroughly spray the tubes containing the blood with sterile alcohol, and place in the laminar flow biosafety cabinet. Spray the first 50 ml conical tube and place in the laminar flow biosafety cabinet. Remove the caps from the blood collection tubes and pour into the 50 ml conical tube. Centrifuge the blood at 3600 rpm and room temperature for 10 minutes. When you stop the centrifuge, do not use the brake.

### Platelet Rich Plasma Isolation

Spray a 20 cc luer lock syringe, a second 50 ml conical tube and a cell extractor with alcohol and transfer to the laminar flow biosafety cabinet. Using the 20 cc syringe and the cell extractor, and, excluding the buffy coat or red blood, collect the Platelet Poor Plasma layer and the Platelet Rich Plasma layer, and add to the second 50 ml conical tube. Centrifuge the tissue at 2400 rpm and room temperature for 10 minutes. Do not use the brake when stopping the centrifuge.

### Platelet Rich Plasma Collection

Using a 3-5 cc syringe and a cell extractor, collect the PRP layer. Remove the cell extractor from the syringe. Transfer the Platelet Rich Plasma layer for immediate use.

### III. Procedure for Cell Counting Procedure Using the Hemocytometer and Viability Calculations

### Materials—

Clean hemocytometer and cover glass; Trypan Blue, 0.4%; conical tubes or equivalent; hand counter; 70% isopropyl alcohol; appropriate disposable tips for pipettors

### Equipment—

Microscope; pipettor; Micropipettor

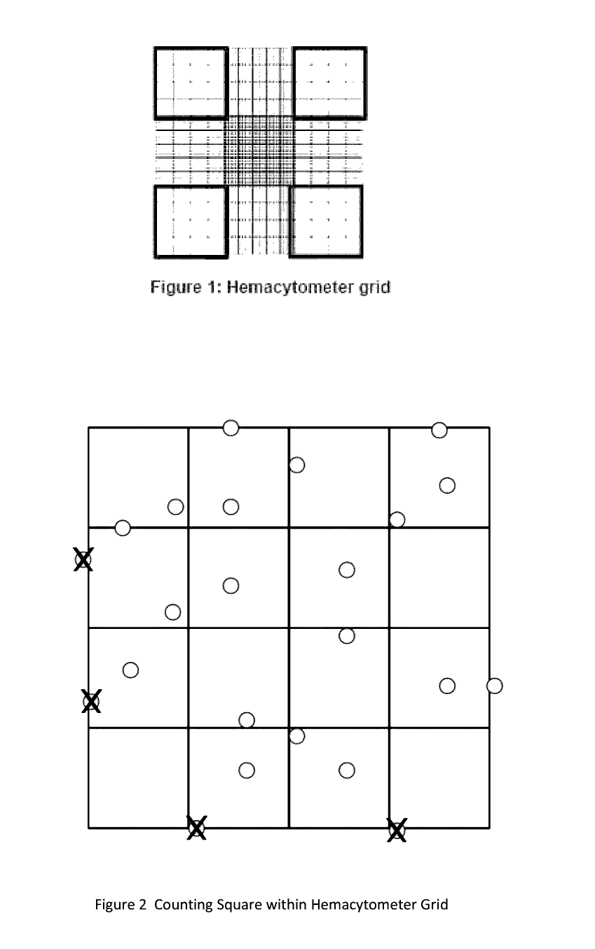

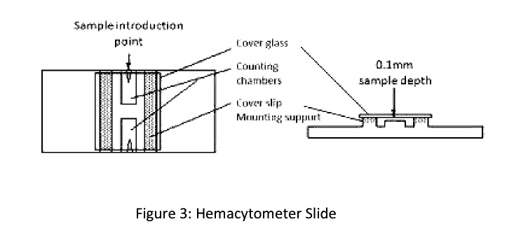

The distinction between living and dead cells can be made using a hemocytometer and the dye exclusion test. The dye used in this procedure is Trypan Blue, a viable stain, which does not penetrate the membrane of viable cells but is absorbed by dead cells which have a deteriorated membrane. The hemocytometer (see FIG. 3 contains of two counting chambers, each of which is divided into nine 1.0 mm squares (See FIG. 1, hemocytometer grid). A cover glass is supported 0.1 mm over these chambers, leaving 0.1 mm of space between the counting chamber and the cover glass (See FIG. 3). Since 1 cm³ is approximately equivalent to 1 mL, the cell concentration per mL is the average count per square, times the dilution factor, times 10⁴.

### Making Working Dilution of Cell Suspension

Mix to make a homogeneous cell suspension and note total volume. Aseptically remove a sample of the suspension and transfer it to a conical tube. If viability count is desired, accurately add the appropriate volume of the 0.4% trypan blue to 20 ul of the cell suspension. The volume will depend on the dilution necessary to have a count of less than 30-150 cells in each square counted.

For 20 ul cell suspension:
i. 10 ul=1:2 dilution
ii. 40 ul=1:3 dilution
iii. 100 ul=1:6 dilution

Growth medium may be added to further dilute solution if necessary, or in lieu of trypan if viability is not required. Mix cell suspension thoroughly using a pipettor just prior to counting.

### 1. Preparation of Hemocytometer

Make sure the hemocytometer and cover glass are clean. If necessary, clean with 70% isopropyl alcohol. Place the cover glass over the hemocytometer chambers as shown in FIG. 3. Using a pipettor, fill both chambers of the hemocytometer with 10 ul each by placing the tip of the pipette at the notch of the hemocytometer, thus allowing the fill to occur by capillary action. Using a microscope with a 10× ocular and a 10× objective, or other appropriate objective to count accurately, count the number of cells in one of the four corners and middle squares of the grid for each hemocytometer chamber (as depicted in FIG. 1). This gives a total of 4 squares counted. (2squares for each of the 2 chambers). Since trypan blue is excluded by the membrane of viable cells but taken up by the nuclei of dead cells, one can distinguish between the blue (non-viable) and the living (viable) cells. Use one button on the hand counter for the living (viable) cells and one button for the non-viable cells. Clumps of cells should never be counted as greater than five even if there are more cells than that in a clump. If over 10% of the cells represent clumps, the entire procedure should be repeated as this is not indicative of a true homogeneous cell distribution. If more than (>)600 cells are present in the count of the four squares, then a greater dilution must be made. If less than (<)50 cells are present, then a lesser dilution must be made. FIG. 2 shows a representative square in a grid of single chamber of a hemocytometer. The standard cell counting convention includes in a count all cells touching the upper or left boundary lines, but none of the cells touching the lower or right boundary lines. In the FIG. 2 diagram, one would count the open figures but not the solid figures.

### Calculations

The viable cell concentration, the total viable cell count, and the cell viability can be calculated using the average number of viable and non-viable cells counted per square, the dilution factor and the total volume of cell suspension.

Viable Cell Concentration (Average # of viable cells per mL):
The cell concentration is the average viable cells per square, times the dilution factor, times 10⁴ (10,000)

Cell Concentration=(Average # of viable cells per square)×(Dilution Factor)×10,000

a. Total Viable Cell Count:
The total viable cell count is the viable cell concentration times the total volume of cell suspension.

Total Viable Cell Count=(Viable Cell Concentration)×(Total Volume of Suspension)

b. Cell Viability:

The cell viability is the average # of viable cells divided by the sum of the average # of viable cells plus average # of dead cells. This number is then multiplied by 100 to get a percent value.

Percent Viability (% Viability)=[(Avg. # of viable cells)÷(Avg. # of viable Cells+Avg. # of dead cells)]×100%

When ranges are used herein for physical properties, ratios of components, cell viability percentages, enzyme activity units, endotoxin levels, and/or cell counts, all combinations and subcombinations of ranges and specific embodiments therein are intended to be included.

The disclosures of each patent, patent application and publication cited or described in this document are hereby incorporated herein by reference, in their entireties.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

When any variable occurs more than one time in any constituent or in any formula, its definition in each occurrence is independent of its definition at every other occurrence. Combinations of substituents and/or variables are permissible only if such combinations result in stable compositions.

It is believed the chemical formulas, abbreviations, and names used herein correctly and accurately reflect the underlying compounds reagents and/or moieties. However, the nature and value of the present invention does not depend upon the theoretical correctness of these formulae, in whole or in part. Thus it is understood that the formulas used herein, as well as the chemical names and/or abbreviations attributed to the correspondingly indicated compounds, are not intended to limit the invention in any way, including restricting it to any specific form or to any specific isomer.

The invention illustratively disclosed herein suitably may be practiced in the absence of any element which is not specifically disclosed herein. The invention illustratively disclosed herein suitably may also be practiced in the absence of any element which is not specifically disclosed herein and that does not materially affect the basic and novel characteristics of the claimed invention.

What is claimed:

1. A stem cell composition comprising:
a population of stem cells obtained from mammalian adipose tissue;
said population of stem cells comprising:
endothelial progenitor cells, red blood cells, fibroblasts, white blood cells, hematopoietic stem cells and mesenchymal stem cells;
wherein the stem cell mixture contains less than about 0.5 EU/cc endotoxin; and
wherein the stem cell mixture is at least about 95% viable as determined by flow cytometry.

2. A stem cell composition according to claim 1, further comprising at least one of:
preadipocytes, pericytes and macrophages.

3. An enzyme composition comprising:
a proteolytic enzyme capable of digesting adipose tissue; and
an enzyme that catalyzes the hydrolytic cleavage of phosphodiester linkages in DNA;
wherein the enzyme composition used for the dissociation of said adipose tissue contains from about 0.25 to about 0.75 Wunsch units or from about 250 to about 750 Collagen Digestion Units and from about 7 to about 15 Kunitz units of DNase I per cubic centimeter of adipose tissue.

4. An enzyme composition according to claim 3, wherein the proteolytic enzyme capable of digesting adipose tissue is a collagenase and the enzyme that catalyzes the hydrolytic cleavage of phosphodiester linkages in DNA is a DNAse.

5. An enzyme composition according to claim 3, wherein the proteolytic enzyme capable of digesting adipose tissue is a collagenase derived from Clostridium histolyticum.

6. A pharmaceutical kit for isolation of stem cells from mammalian adipose tissue comprising a container having an effective amount of an adipose digestion enzyme composition according to claim 3.

7. A kit according to claim 6, further comprising conventional stem cell isolation kit components.

8. A kit according to claim 7, wherein said conventional stem cell isolation kit components include at least one of the following: a syringe, cell strainer, syringe filter, cell wash solution, cell extractor, conical tube, cell wash bag, and sterile container with lid.

9. A kit according to claim 6, further comprising instructions for isolating stem cells from mammalian adipose tissue.

10. A method for isolating stem cells from mammalian adipose tissue comprising the steps of:
contacting mammalian adipose tissue with an enzyme composition according to claim 3 for a time and under conditions effective to digest the adipose tissue; and
isolating the cells from the enzyme digested adipose tissue;
wherein the adipose tissue was extracted from a mammal.

11. A method according to claim 10, further comprising a mammalian adipose tissue extraction process comprising the steps of:
inserting a cannula into a subcutaneous space containing adipocytes in said mammal;
wherein the cannula is attached to a syringe;
aspirating said subcutaneous space for a time and under conditions effective to transfer said adipocytes containing an amount of ruptured adipocytes or blood components from the subcutaneous space via cannula to the syringe; and
rinsing the adipocytes for a time and under conditions effective to reduce the amount of ruptured adipocytes or blood components contained in the transferred adipocytes and to provide the extracted mammalian adipose tissue.

12. A stem cell composition prepared by the process according to claim 10.

13. A method for treating a medical condition amenable to treatment with stem cells comprising the steps of:
isolating stem cells from adipose tissue of a patient in need of treatment for a medical condition amenable to treatment with stem cells;
providing a stem cell composition according to claim 1 comprising said isolated stem cells; and
reimplanting an effective amount of said stem cell composition into an anatomical region of-said patient, wherein said region is affected with said medical condition.

14. A method for transferring autologous fat for cosmetic anatomical improvement, comprising the steps of:
isolating stem cells from adipose tissue at a first anatomical location of a patient in need of cosmetic anatomical improvement;
providing a stem cell composition according to claim 1 comprising said isolated stem cells; and
reimplanting an effective amount of said stem cell composition at a second anatomical location of said patient, wherein said second location requires said cosmetic anatomical improvement.

15. A method for rejuvenating skin or improving the appearance of skin in a mammal, comprising the steps of:
isolating stem cells from adipose tissue in a patient in need of skin appearance improvement or rejuvenation;
providing a stem cell composition according to claim 1 comprising said isolated stem cells; and
reimplanting an effective amount of said stem cell composition into an epidermal anatomical region of said patient, wherein said region requires said skin appearance improvement or rejuvenation.

16. A method for modifying the shape of a mammalian anatomical region comprising the steps of:
isolating stem cells from adipose tissue of a patient in need of an anatomical region shape modification;
providing a stem cell composition according to claim 1 comprising said isolated stem cells; and
reimplanting an effective amount of said stem cell composition into an anatomical region of said patient, wherein said region requires shape modification.

17. An enzyme composition comprising:
a proteolytic enzyme capable of digesting human adipose tissue; and
an enzyme that catalyzes the hydrolytic cleavage of phosphodiester linkages in DNA;
wherein said enzyme composition is sufficient to digest said human adipose tissue, and provide a stem cell composition containing at least about 2.0E+05 cells per cc LPA.

18. The enzyme composition according to claim 17, wherein the stem cell composition is at least about 95% viable as determined by flow cytometry.

19. The enzyme composition according to claim 17, wherein the stem cell composition contains from about 20 to about 25% hematopoietic stem cells and from about 30 to about 35% mesenchymal stem cells as determined by flow cytometry.

20. A pharmaceutical composition comprising:
a stem cell composition according to claim 1; and
platelet rich plasma.
